(19) United States
(12) Patent Application Publication
(10) Pub. No.: US 2005/0267413 A1
(43) Pub. Date: Dec. 1, 2005

(54) Flow monitoring devices and methods of use

(76) Inventors: Jong H. Wang, Rancho Palos Verdes, CA (US); Siddharth Desai, Mission Viejo, CA (US)

Correspondence Address: BAXTER HEALTHCARE CORPORATION, ONE BAXTER PARKWAY, DF2-2E, DEERFIELD, IL, 60015, US

(21) Appl. No.: 10/853,916

(22) Filed: May 26, 2004

(51) Int. Cl. A61M 37/00

(52) U.S. Cl. 604/131; 604/67; 604/246

(57) ABSTRACT

The present invention discloses a number of devices and methods for monitoring a flow within a flow circuit. In one embodiment, a flow indicator is disclosed and includes a flow indicator device having a body, the body having first conduit port and a second conduit port formed thereon, the second conduit port in communication with an ambient environment, at least one indicator conduit in fluid communication with an infusion circuit and the first conduit port, a sensing device positioned within the body and in fluid communication with the first and second conduit ports, and at least one indicator positioned on the body and in communication with the sensing device.

### BACKGROUND

Infusion therapy is accomplished by administering various fluid formulations to patients. Commonly, the infusion of fluids into the human body is usually accomplished by means of a source of fluid, an infusion circuit and means for forcing the fluid through the circuit. In some embodiments the infusion assembly also includes a monitoring apparatus which monitors the rate of flow of fluid through the infusion circuit.

In a hospital environment, intravenous infusion is accomplished by using either a gravity infusion methods or a pump driven device to force the fluid through the circuit. Such pump driven devices can include electromechanical devices and infuser devices. Gravity infusion methods typically utilize administration sets having drip chambers with drop formers which enable flow to be set and monitored by the counting of drops exiting the drop former over a set period of time. However, it is difficult to accurately set and monitor flow rates, particularly at low flow rates. Electromechanical pump driven devices allow the flow rate of fluid to be set with great precision and typically include various fluid flow displays and alarms which indicate flow discontinuities. However, such devices are expensive and are heavy and bulky hinder a patient's freedom of movement.

Often, the infusion of medicaments or other therapeutic agents may be required over long periods of time with a patient that is ambulatory. In such situations, infusion therapy should be accomplished without requiring a patient to remain in one location by using a portable or ambulatory infuser. Various ambulatory electronic pumps as well as disposable mechanical devices are known. Electronic pumps however, are expensive and must be returned to the health care center for reuse.

Disposable devices are suitable for providing a low cost ambulatory infusion therapy. Such devices include elastomeric infusers and spring biased infusers. Disposable devices of this type often operate at extremely low flow rates as slow as 0.5 ml/hr. Flow indications are provided either by volume gradations or a dipstick-like device. However, due to the low flow rates, resulting in small changes in volume, it may take a long time, up to 10-20 hours or more, to observe a volume change. As a result, the lack of a reliable and quick way to ascertain flow continuity is a shortcoming of currently available infusion devices for home use and is a known cause of anxiety in home patients. However, any flow indicating devices may not appreciably increase the cost of such disposable devices.

In light of the foregoing, there is presently a need for a ambulatory infusion device which includes an inexpensive flow monitoring device capable of quickly and reliably ascertaining data regarding a flow from an infusion device to a patient and notifying the patient of such data. Moreover, there is a further need for the flow monitoring device to be utilized with a disposable infusion device.

### BRIEF SUMMARY

The embodiments of the flow monitoring devices disclosed herein enable a user to monitor the flow of material through a flow circuit of an ambulatory infusion device without the problems associated with prior art devices.

In one embodiment, the present application discloses an ambulatory infusion system including a flow indicator device. The flow indicator device has a body, the body having a first port and a second port formed thereon, the second conduit port in sensory communication with an ambient environment, the first conduit port in sensory communication with infusion fluid flowing through an infusion circuit. A sensing device is positioned within the body and in sensory communication with the first and second conduit ports, and the device includes at least one indicator positioned on the body and communication with the sensing device.

In another embodiment, the present application discloses an ambulatory infusion system that includes a flow indicator device having a body, the body having a first conduit port and a second conduit port formed thereon, the second conduit port in fluid communication with an ambient environment, at least one indicator conduit in fluid communication with both an infusion circuit and the first conduit port, a sensing device positioned within the body and in fluid communication with the first and second conduit ports, a flow indicator positioned on the body and in communication with the sensing device and a no-flow indicator positioned on the body and in communication with the sensing device.

In an additional embodiment, the present application discloses an ambulatory infusion device including a flow circuit and a flow meter for measuring and indicating flow in the flow circuit. The flow circuit including a flow restrictor, an upstream flow conduit and a downstream flow conduit. The flow meter includes a flow meter body having at least one display device thereon, an upstream flow port formed on the flow meter body and in sensory communication with an upstream flow conduit, a downstream flow port formed on the flow meter body and in sensory communication with a downstream flow conduit, and a sensing device positioned within the flow meter body and in fluid communication with the upstream flow port and the downstream flow port, the sensing device in communication with the display device.

In still another embodiment, the present application discloses a flow meter comprising a flow meter body having at least one display device disposed thereon, an upstream flow port formed on the flow meter body and in fluid communication with an upstream flow conduit, the upstream flow conduit in communication with a flow circuit upstream of a flow restrictor, a downstream flow port formed on the flow meter body and in fluid communication with a downstream flow conduit, the downstream flow conduit in communication with a flow circuit downstream of the flow restrictor, a sensor housing positioned within the flow meter body and defining a sensor receiving cavity, the sensor receiving cavity in fluid communication with the upstream flow port and the downstream flow port, and a sensor positioned within the sensor receiving cavity and in communication with the display device, the sensor configured to compare a pressure measured upstream of the flow restrictor to a pressure measure downstream of the flow restrictor.

In still another embodiment, the present application discloses a flow meter and indicator and includes a body having at least one display device and at least one indicator disposed thereon, a upstream flow port formed on the body and in fluid communication with an upstream flow conduit, the upstream flow conduit in communication with a flow circuit upstream of a flow restrictor, a downstream flow port formed on the body and in fluid communication with an downstream flow conduit, the downstream flow conduit in communication with a flow circuit downstream of the flow restrictor, and a sensing device positioned within the flow indicator body and in fluid communication with the upstream flow port, downstream flow port, the sensing device in communication with the display device and the indicator. In a further embodiment, the body includes means for recording the flow rate and corresponding time and calculating other information from these measurements. Such other information including the volume infused.

The present application also discloses a sensor device having a sensor housing formed by a first body and a second body, the sensor housing having a sensor receiving cavity formed therein, a first passage formed in the sensor housing and in communication with the sensor receiving cavity and a first conduit port, a second passage formed in the sensor housing and in communication with the sensor receiving cavity and a second conduit port, and a sensor positioned within the sensor receiving cavity and configured to compare a pressure from a first conduit port to a pressure from the second conduit port.

The present application also discloses a method of determining if a flow exists within a flow circuit and includes sensing a pressure within a flow circuit with a sensor, sensing an ambient pressure within a environment with the sensor, and comparing the pressure within the flow circuit to the ambient pressure.

In addition, the present application discloses a method of measuring a flow rate through a flow circuit having a flow restrictor positioned therein and includes sensing a pressure upstream of the flow restrictor with a sensor, sensing a pressure downstream of the flow pressure with the sensor, comparing the pressure upstream of the flow restrictor to the pressure downstream of the flow restrictor, and calculating a flow rate based on a pressure differential between the upstream flow pressure and the downstream flow pressure. In a further method the flow rate calculation is utilized in calculating the volume delivered or infused and volume remaining.

In still another embodiment, the present application discloses a method of indicating a flow and measuring a flow rate through a flow circuit having a flow restrictor coupled thereto. More specifically, the present application discloses sensing a pressure upstream of the flow restrictor with a sensor, sensing a pressure downstream of the flow pressure with the sensor, comparing the pressure upstream of the flow restrictor to the pressure downstream of the flow restrictor, and calculating a flow rate based on a pressure differential between the upstream flow pressure and the downstream flow pressure.

Other objects, features, and advantages of the embodiments of the flow monitoring devices disclosed herein will become apparent from a consideration of the following detailed description.

### BRIEF DESCRIPTION OF THE DRAWINGS

The flow monitoring devices of the present application will be explained in more detail by way of the accompanying drawings, wherein:

FIG. 1 shows a perspective view of an infusion circuit having an embodiment of a flow indicator coupled thereto;

FIG. 2 shows a perspective view of an embodiment of a flow indicator;

FIG. 3 shows a block diagram of an embodiment of a control circuit useful in a flow indicator;

FIG. 4A shows a schematic diagram of the embodiment of the control circuit shown in FIG. 3;

FIG. 4B shows a schematic diagram of an alternate embodiment of the control circuit shown in FIG. 3;

FIG. 5 shows a perspective view of an embodiment of a sensor section and housing for use in a flow indicator;

FIG. 6 shows a side view of the embodiment of the sensor section and housing shown in FIG. 5;

FIG. 7 shows an exploded view of the embodiment of the sensor section and housing shown in FIG. 5;

FIG. 8 shows a cross sectional view of the embodiment of the sensor section and housing as taken along the lines 8-8 shown in FIG. 5;

FIG. 9 shows a perspective view of an infusion circuit having an embodiment of a flow meter coupled thereto;

FIG. 10 shows a perspective view of an embodiment of a flow meter;

FIG. 11 shows a block diagram of an embodiment of a control circuit useful in a flow meter;

FIG. 12A shows a schematic diagram of the embodiment of the control circuit shown in FIG. 11;

FIG. 12B shows a schematic diagram of an alternate embodiment of the control circuit shown in FIG. 11;

FIG. 13 shows a perspective view of an embodiment of a sensor section and housing for use in a flow meter;

FIG. 14 shows a side view of the embodiment of the sensor section and housing shown in FIG. 13;

FIG. 15 shows an exploded view of the embodiment of the sensor section and housing shown in FIG. 13;

FIG. 16 shows a cross sectional view of the embodiment of the sensor section and housing as taken along the lines 16-16 shown in FIG. 13;

FIG. 17 shows a perspective view of an embodiment of a flow meter and indicator; and

FIG. 18 shows a block diagram of an embodiment of a control circuit useful in a flow meter.

### DETAILED DESCRIPTION

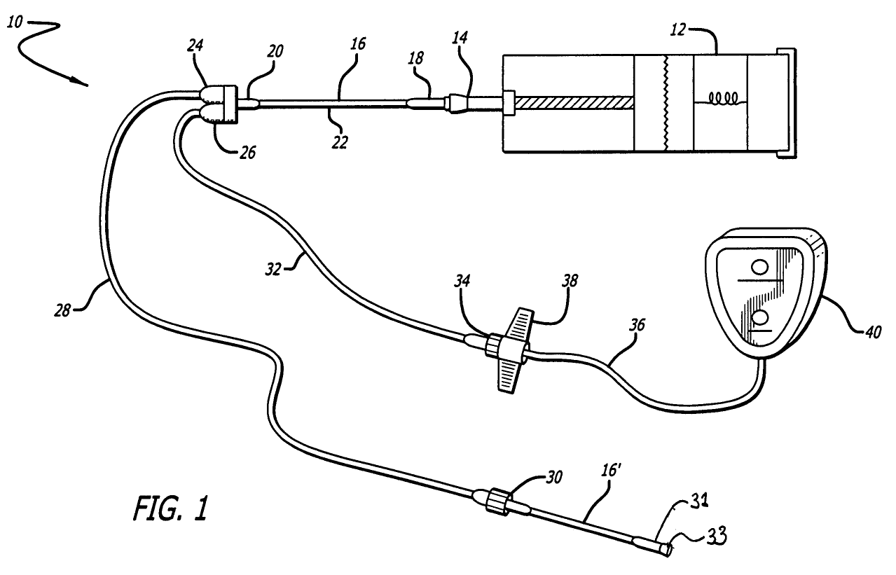

FIG. 1 shows an ambulatory system for infusing or otherwise delivering medicament or other therapeutic agents to a patient. As shown, an infusion circuit 10 is attached and in fluid communication with an infusion device 12 for example a pump. The circuit 10 may be attached to the device 12 with either a permanent or removable connector such as a luer connection assembly (not shown). The infusion circuit 10 defines a passageway for fluid expelled by the infusion device 12. The device 12 has an output 14 coupled to a flow restrictor 16 forming a component of the infusion circuit 10. In the illustrated embodiment, the infusion device 12 comprises a device having an elastomeric bladder to hold and pressurize the fluid for delivery. Optionally, any of a variety of infusion devices may be used with the present system.

Referring again to FIG. 1, the flow restrictor 16 comprises a flow restrictor inlet 18 coupled to the output 14 of the infusion device 12, and a flow restrictor outlet 20. A restrictor body 22 is positioned between the flow restrictor inlet 18 and the flow restrictor outlet 20. In one embodiment, the transverse dimension of the flow restrictor body 22 is constant. For example, the flow restrictor body 22 defines a passageway having a constant diameter along the length thereof. Several embodiments of flow restrictors 16 are contemplated such as glass capillaries, micro-tubing or other restrictions.

As illustrated in FIG. 1, the flow restrictor outlet 20 includes an infusion conduit port 24 and a monitor conduit port 26. The infusion conduit port 24 is sized and configured to receive an infusion conduit 28 therein or have one coupled thereto. The infusion conduit 28 further includes a distal portion having an infusion connector 30. The infusion connector 30 is connected to a second flow restrictor 16′ such that fluid communication is established between first restrictor 16 and second restrictor 16. The flow restrictor 16′ is preferably of the same construction as the flow restrictor 16 although other restrictor designs are contemplated. A distal end 31 of the second flow restrictor 16′ is configured to couple to a variety of infusion devices such as, without limitation, catheters, implantable ports, intravenous delivery devices, shunts, or other mechanisms capable of delivering medicament to a patient. Prior to use, a removable tip cap 33 closes off the distal end 31.

Referring again to FIG. 1, the monitor conduit port 26 is configured to couple to or receive a monitor conduit 32 therein. The monitor conduit 32 may include a monitor coupler 34 attached thereto. The monitor coupler 34 is configured to releasably couple to an indicator conduit 36 through a connector 38. The indicator conduit 36 is coupled to a flow indicator 40 with a fluid tight sealed connection.

The flow restrictor 16, infusion conduit 28, the monitor conduit 32, and/or the indicator conduit 36 may be manufactured in any variety of sizes and lengths as desired. In the preferred embodiment these are manufactured of medical grade tubing. Further, the various connectors and couplers illustrated in FIG. 1 may be configured to detachably or nondetachably couple the various elements illustrated in FIG. 1 together. In the preferred embodiment the infusion circuit is sterilized so that there is a sterile passageway for fluid expelled from the infusion device 12. Those in the art know various methods of sterilization for such devices.

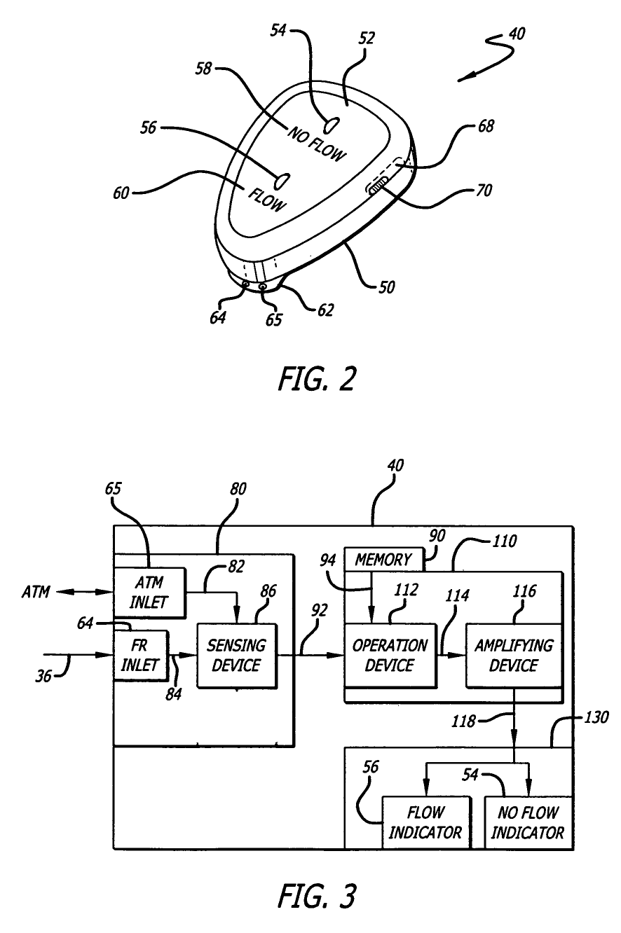

FIG. 2 shows an embodiment of a flow indicator 40 shown in the infusion circuit 10 of FIG. 1. As shown, the flow indicator 40 includes a body 50 defining a face 52 having a first indicator 54 and a second indicator 56 positioned thereon. In the illustrated embodiment, a first legend 58 is positioned proximate to the first indicator 54, and a second legend 60 is positioned proximate to the second indicator 56. Optionally, the flow indicator 40 may be manufactured without the first legend 58, the second legend 60, or both. The body 50 of the flow indicator 40 further includes a back plate 62 having at least one conduit port 64 formed thereon. In the illustrated embodiment, the back plate 62 includes a first conduit port 64 and a second conduit port 65 formed thereon. In the illustrated embodiment, an activation switch 70 is positioned within a switch recess 68 formed on the body 50 of the flow indicator 40. In an alternate embodiment, the activation switch 70 and the switch recess 68 may be positioned anywhere upon the body 50 of the flow indicator 40. Optionally, the flow indicator 40 may be manufactured without the activation switch 70 and the switch recess 68.

In the embodiment illustrated in FIG. 2, the first indicator 54 comprises a red light emitting diode while the second indicator 60 comprises a green light emitting diode. Optionally, any number, size, or color of light emitting diodes may be used as the first indicator 54, second indicator 56 or both. Moreover, any variety of indicators may be used with the flow indicator 40, including, incandescent bulbs, fuses, switches, liquid crystal displays, plasma displays, integrated circuit displays, or other information display devices. Moreover, the flow indicator 40 may include an audible alarm either alone or in combination with the visual indications.

FIG. 3 shows a block diagram of an embodiment of a control circuit for use within the flow indicator 40. As shown, the first conduit port 64 is coupled to the indicator conduit 36 which is in communication with the infusion circuit 10 downstream of the flow restrictor 16 and upstream of the second flow restrictor 16′ (See FIG. 1). The indicator conduit 36 is preferably connected to the first conduit port 64 in an airtight connection. The first conduit port 64 is in communication with and provides information 84 to the sensing device 86 positioned within a sensor section and housing 80. Further, a second conduit port 65 may be in communication with the ambient environment which provides a reference pressure. As such, the second conduit port 65 provides information 82, such as the pressure of the ambient environment, to the sensing device 86.

Referring again to FIG. 3, the sensing device 86 provides information 92 to the operational device 112 of the processing section 110. In one embodiment, the operational device 112 comprises an operational amplifier that is in communication with the sensing device 86 of the sensor section and housing 80. In addition, the processing section 110 may include a memory device 90 positioned therein. The memory device 90 may be configured to receive and store flow information received from the operational device 112 therein. In addition, the memory device 90 may be capable of storing a library of data relating to the flow characteristics of various medicaments or therapeutic agents. Exemplary memory devices include, without limitation, erasable programmable read-only memory devices (EPROMS). The operational device 112 processes the input information and provides a process signal 114 to the amplifying device 116. The amplifying device 116 amplifies the process signal 114 and provides an amplified signal 118 to the indicator section 130.

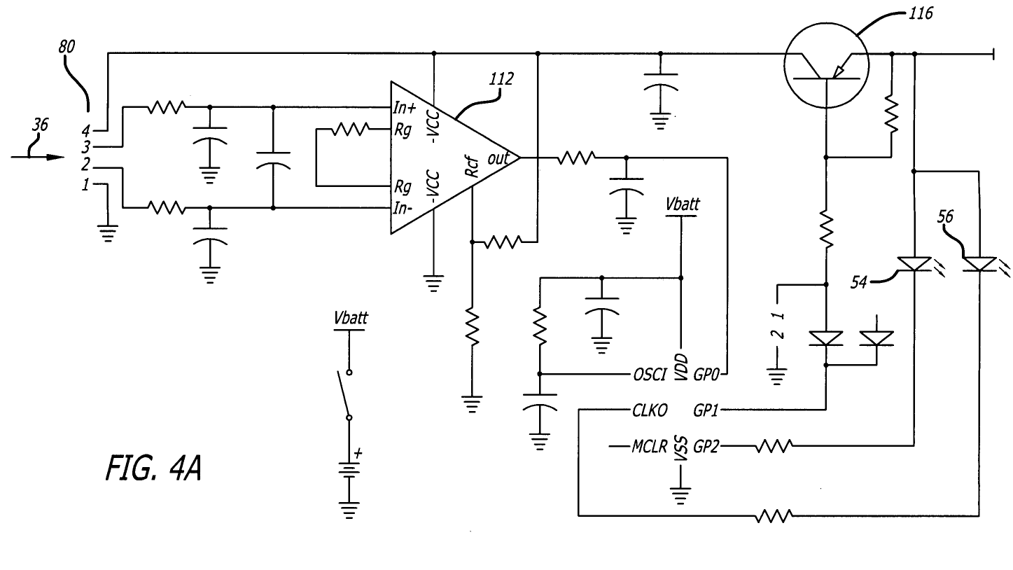

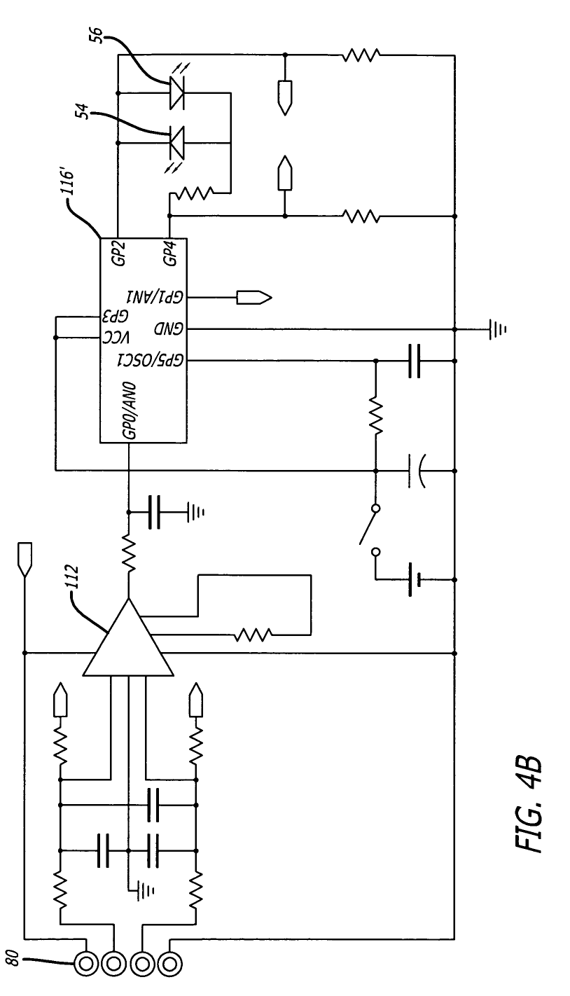

In one embodiment, an amplifying device 116 comprises a PNP transistor. FIG. 4A shows a schematic diagram of an embodiment of a pressure watch circuit for use within the flow indicator 40 using a transistor as an amplifying device 116. Optionally, any number or variety of amplifying devices may be used within the processing section 110. For example, the amplifying device 116 may comprise a programmable integrated circuit device. FIG. 4B shows an alternate embodiment of a pressure watch circuit using a programmable integrated circuit device 116′ as an amplifying device.

The indicator section 130 of the flow indicator 40 includes a first indicator 54 and at least a second indicator 56. As shown, one indicator may be used to indicate normal flow operation, while the other indicator may be used to indicate a disruption of the flow process. Other types of indicators including audible indicators are also contemplated.

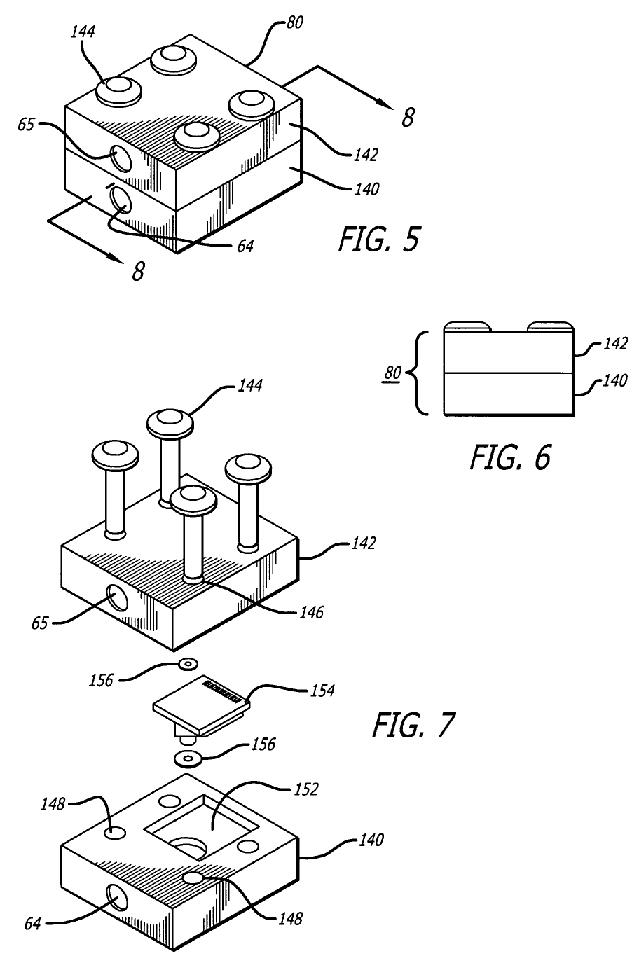

FIGS. 5-8 show an embodiment of a sensor section and housing 80 for use in a flow indicator 40. As shown, the sensor housing 80 comprises a first housing body 140 and a second housing body 142 which cooperatively form the sensor housing 80. In the illustrated embodiment, one or more fastening devices 144 may be used to couple the second housing body 142 to the first housing body 140. Optionally, any of a variety of fastening devices 144 may be used to couple the housing bodies 140 and 142, including screws, bolts, pins, lock members, adhesives, latches, or other fastening devices. In an alternate embodiment, the housing bodies 140 and 142 may be bonded to each other using a bonding agent or ultrasound bonding techniques. In the illustrated embodiment, the second housing body 142 includes one or more fastener passages 146 formed thereon configured to receive the fastening devices 144 therethrough. Similarly, the first housing body 140 includes one or more fastener receiving ports 148 thereon configured to receive and engage a fastening device 144 therein.

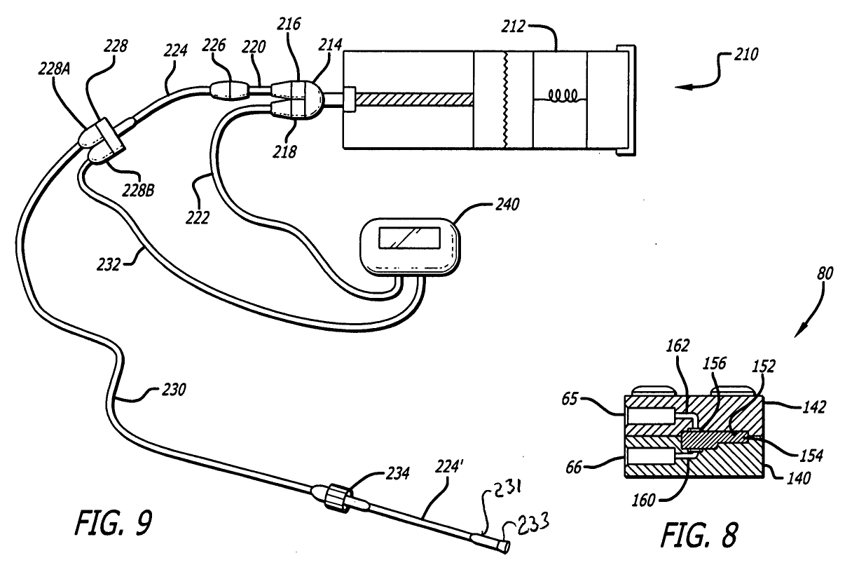

As shown in FIG. 7, the first housing body 140 further defines a sensor receiving cavity 152 therein. As shown in FIG. 8, the sensor receiving cavity 152 is in communication with the first conduit port 64 through a first passage 160. Similarly, the second housing body 142 defines the sensor receiving cavity 152 which is in communication with the second conduit port 65 through a second passage 162. A pressure sensor or transducer 154 is positioned within the sensor receiving cavity 152 formed by the first and second housing bodies 140, 142. As such, the sensor 154 is in communication with the first conduit port 64 and the second conduit port 65 and is particularly designed to sense the pressure differential between the pressures present at the first and second conduit ports. In one embodiment, the pressure sensor 154 comprises a solid state pressure sensor. For example, the pressure sensor 154 may comprise a solid state piezoresistive pressure sensing device. Optionally, one or more seal devices 156 may be positioned within or proximate to the first passage 160, the second passage 162, or both, thereby sealing the sensor 154 within the sensor housing 80.

As the infusion circuit 10 is used to introduce fluids intravenously into a patient, the device should be sterilized and properly packaged. All passageways through which fluid flows or is exposed to should be sterilized and maintained in a sterile manner prior to use.

During use, the infusion device 12 is filled with the fluid to be infused. The output 14 of the device 12 may then be connected to the first flow restrictor 16. The tip cap 33 is then removed and the infusion circuit 10 is primed. In priming, the infusion device 12 pressurizes the fluid and the fluid flows from the output 14 of the device along a passageway formed by the first flow restrictor 16, infusion conduit port 24, infusion conduit 28, connector 30, second flow restrictor 16′, and the fluid exits from the distal end 31 of the restrictor. The tip cap 35 may be replaced until the infusion circuit 10 is ready to be employed or the distal end 31 may be connected to a device for infusing fluids into a patient.

When the fluid flows past the monitor conduit port 26 and through the infusion conduit port 25, and with the indicator conduit 36 in airtight engagement with the flow indicator 40 a sealed column of air is formed in the monitor conduit 28 and indicator conduit 36. The pressurized fluid will travel partially up the monitor conduit 28 compressing the air column in the monitor conduit and indicator conduit 36 until the air pressure reaches equilibrium with the pressure of the fluid. The pressure of this air column, and therefore the pressure of the fluid flowing past the monitor conduit port 26, is sensed by the first conduit port 64 such that the first conduit port is in sensory communication with the pressure of the fluid at the monitor conduit port 26.

The sensor 154 (FIG. 8) compares the pressure measured within the infusion circuit 10 as received through the first conduit port 64 with the ambient pressure, typically about 14.7 psia which is equal to 0.0 psig, as measured through the second conduit port 65. During a normal infusion process, the pressure within the infusion circuit 10 drops along across the flow restrictors 16, 16′. At the outlet 14 the pressure corresponds to the pressure of the infusion device 12. In a preferred embodiment of the infusion device, the outlet pressure may be around 8 psig. In the preferred embodiment, immediately downstream of the restrictor 16 at the flow restrictor outlet 20 the pressure is equal to about 4 psig. Downstream of the flow restrictor 16′ the pressure will be slightly greater than a patient's vein pressure, which is about 0.04-0.1 psig (2 to 5 mmHg).

The pressure drops across the flow restrictors 16, 16′ is dependent on fluid flowing through the restrictors, thus a variation of the measured pressure from about 18.7 psi may be indicative of a blockage within the infusion circuit 10. For example, a blockage upstream of a flow restrictor outlet 20 of the flow restrictor 16 would result in the sensor 154 measuring less than 4.0 psig. and in proximity to the vein pressure of the patient. As such, a pressure measurement of between about 0.04-0.1 psig and about 4 psi would indicate an upstream blockage.

In contrast, a blockage downstream of the flow restrictor outlet 20 would result in the sensor 154 measuring a pressure greater than about 4 psig and approaching or equal to the pressure of the fluid in the infusing device 12. As such, a pressure measurement of between about 4.0 psig and about 8 psig would indicate a downstream blockage. As a result, the flow indicator 40 is capable of detecting if a flow is present within the flow circuit 10. As such, the flow indicator 10 is capable of indicating the presence of flow even if there are very low flows within the flow circuit 10. For example, in one embodiment, the flow indicator 40 is capable of detecting very low flow rates of about 0.1 ml/hr to about 5 ml/hr.

Referring again to FIGS. 2 though 7, during normal infusion processes the sensor 154 detects a pressure gradient of about 4 psi between the infusion circuit 10 and the ambient environment thereby illuminating the second indicator 56 on the face 52 of the flow indicator 40, while the first indicator 54 is not illuminated. When the pressure gradient between the infusion circuit 10 and the ambient environment derivates from about 4 psi the first indicator 54 of the flow indicator 40 is illuminated and the second indicator 56 is not illuminated. In one embodiment the first indicator 54, second indicator 56, are configured to remain illuminated during their respective flow conditions. Optionally, the first indicator 54, or second indicator 56 are configured to blink intermittently to indicate flow conditions. In addition an audible alarm may sound.

FIG. 9 shows an alternate embodiment of an infusion circuit which is particularly suitable for not only indicating flow but also measuring the flow. As shown, the infusion circuit 210 is connected and in fluid communication with an infusion device 212 such as a pump. The device 212 may be connected to an output 214 of the infusion circuit 210. The infusion circuit 210 defines a passageway for fluid expelled by the infusion device 212.

The output 214 of the infusion device 212 includes a first port 216 and a second port 218. The first port 216 is coupled to a flow path 220 having a flow restrictor 224 coupled thereto through a coupler 226. The second port 218 is coupled to an upstream flow conduit 222 that is connected to a flow meter 240. Referring again to FIG. 9, the downstream end of the flow restrictor 224 includes a splitter 228 coupled thereto. The splitter 228 includes a first outlet 228A which is connected to an infusion conduit 230 terminating with an infusion coupler 234. In addition, the splitter 228 includes a second outlet 228B having downstream flow conduit 232 coupled thereto. The downstream flow conduit 232 is coupled to the flow meter 240. Like the previous embodiment, in one embodiment the flow restrictor 224 defines a passageway having a constant transverse dimension. Like the previous embodiment, the infusion circuit 210 may include additional flow restrictors. As shown, a second flow restrictor 224′ is coupled to the infusion circuit 210. A distal end 231 of the second flow restrictor 224′ is configured to couple to a variety of infusion devices such as, without limitation, catheters, implantable ports, intravenous delivery devices, shunts, or other mechanisms capable of delivering medicament to a patient. Prior to use, a removable tip cap 233 closes off the distal end 231.

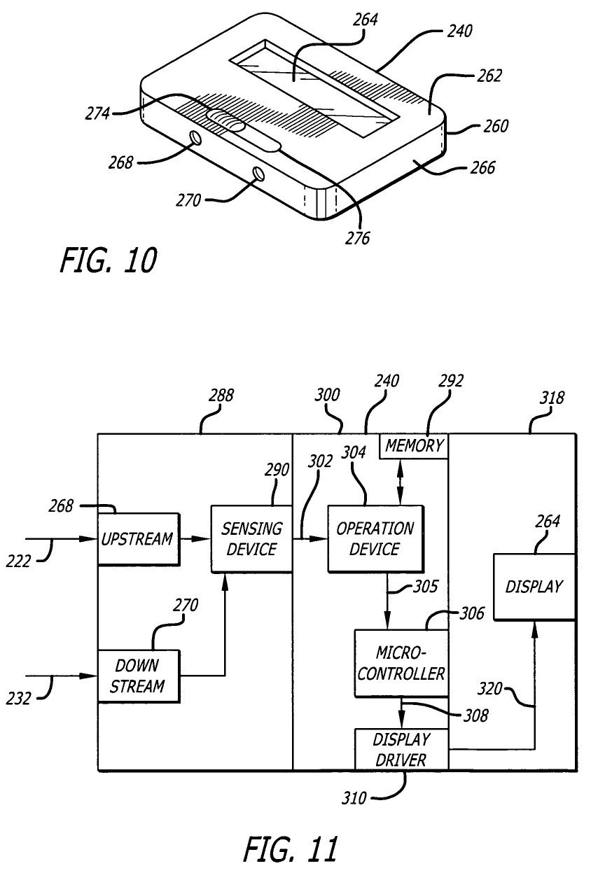

FIG. 10 shows an embodiment of a flow meter 240. The flow meter 240 includes a body 260 having a face 262. The face 262 further disposes an information display 264 thereon. Exemplary information displays include, for example, liquid crystal display devices, plasma display devices, or the like. The body 260 of the flow meter 240 further includes a sidewall 266 having an upstream flow port 268 and a downstream flow port thereon. Optionally, the flow meter 240 may include an activation switch 274 positioned within a switch recess 276.

FIG. 11 shows a block diagram of an exemplary flow meter circuit for use in the flow meter 240. As shown, the upstream flow conduit 222 is coupled to the upstream flow port 268 in an airtight engagement. The upstream flow conduit 222 provides upstream pressure or flow information to the sensing device 290 of the sensor section and housing 288. Similarly, the downstream flow conduit 232 is coupled to the downstream flow port 270 in an airtight engagement. The downstream flow conduit 232 provides downstream pressure or flow information to the sensing device 290. The sensing device 290 of the sensing section and housing 288 is in communication with an operational device 304 positioned within the processing section 300 via a conduit 302. The operational device 304 is in communication with a memory device 292 positioned within the processing section 300 and may be configured to receive and store flow information received from the sensing device. In addition, the memory device 292 may be capable of storing a library of data relating to the flow characteristics of various medicaments or therapeutic agents. In one embodiment, the operational device 304 may comprise a microprocessor and may be include a timing device, such as an internal clock device, therein. As such, the flow meter 240 may be capable of measuring total flow volume through the flow circuit 210 by measuring the flow rate at various points of time and calculating the volume of flow which has occurred over set periods of time. Furthermore, if the initial volume of the infusion device 212 is proved to the flow meter, the volume remaining in the device 212 may be displayed if desired by the user.

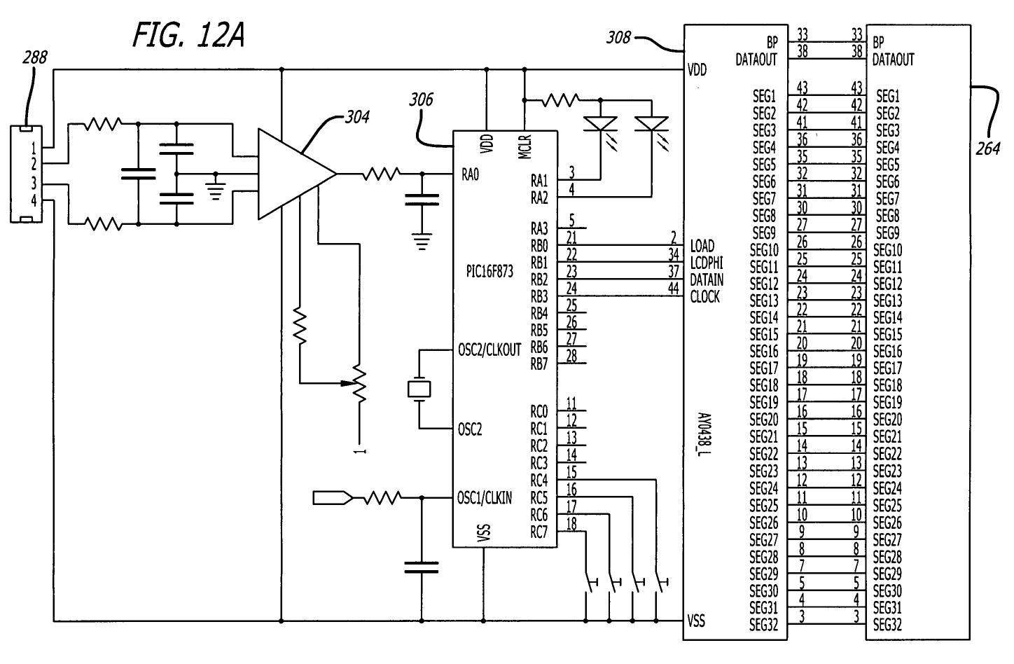

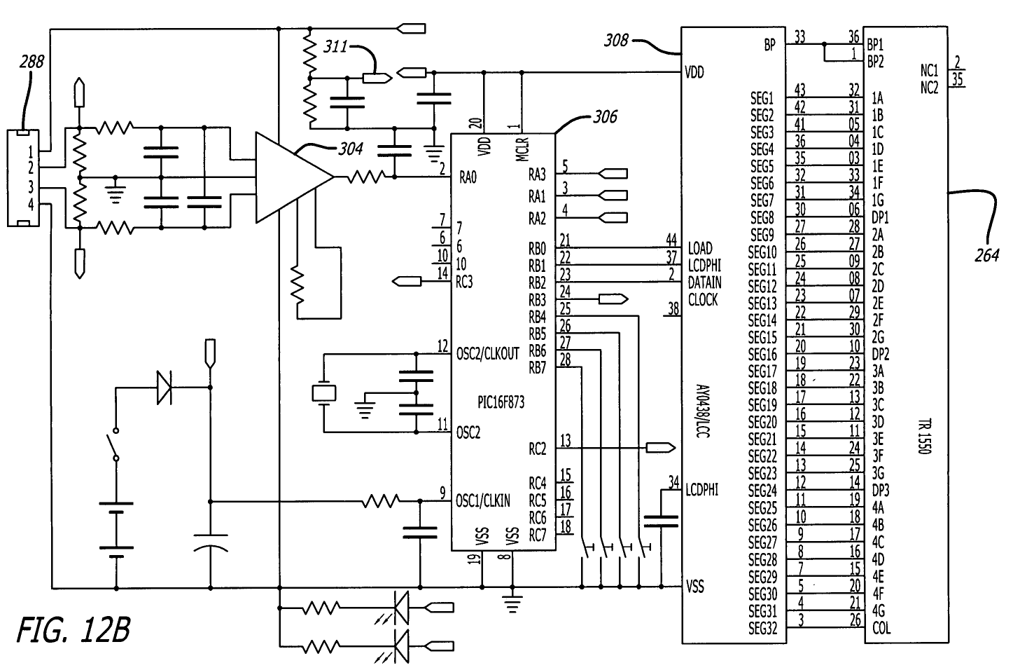

Referring again to FIG. 11, the operational device 304 is also in communication through conduit 305 with a microcontroller positioned within the processing section 300. The operational device 304 and microcontroller 306 receive sensing information from the sensor device 290, processes the information and forward instructions to a display driver 310 via a conduit 308. The display driver 310 of the processing section 300 processes the information received from the microcontroller 306 and provides a driver signal to a display 264 within the display section 318 via a conduit 320. FIG. 12A shows a schematic diagram of an exemplary infusion processing control circuit for use in a flow meter 240.

Optionally, the flow meter 240 may include one or more additional sensing devices. For example, the flow meter 240 may include a temperature sensing device configured to measure the temperature of a fluid within the flow circuit 210. FIG. 12B shows a schematic diagram of an exemplary infusion processing control circuit for use in the flow meter 240. As shown, a temperature sensing device 311 is included within the processing circuit of the flow meter 240. The temperature sensing device 311 may be in communication with the sensing section and housing 288, the operational device 304, or both. (See FIG. 11) Any variety of temperature measuring devices may be used include, without limitation, thermocouples, thermistors, and the like.

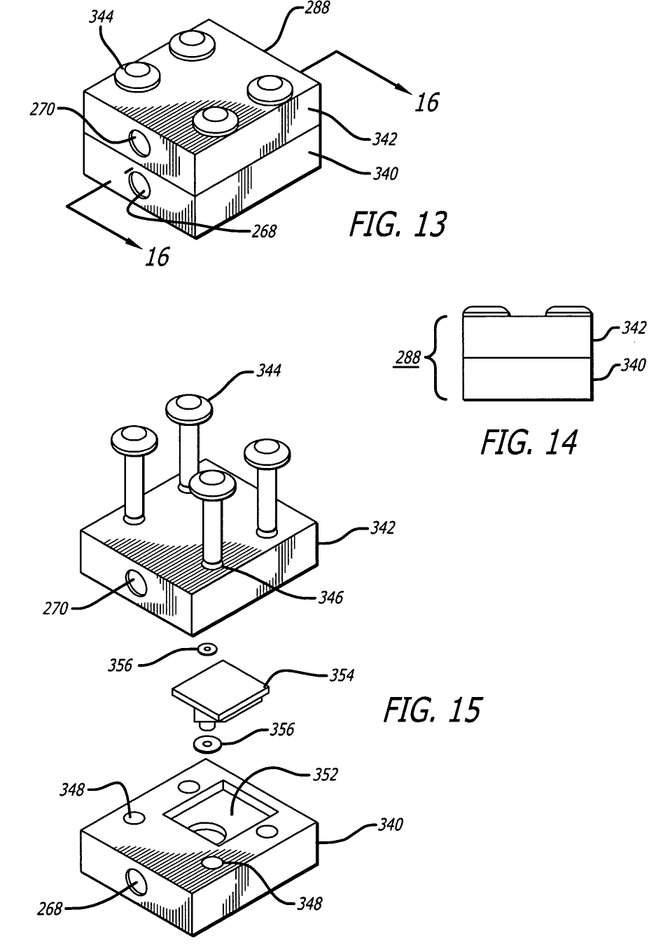

Like the previous embodiment, the flow meter 240 utilizes a sensor section and housing 288 similar to the sensor section and housing 80 illustrated in FIGS. 5 through 8. As shown in FIGS. 13-16, the sensor housing 288 comprises a first body 340 and a second body 342 which is coupled to the first body 340 with one or more fastening devices 344. As shown in FIG. 15, the second body 342 includes one or more fastener passages 346 formed thereon and configured to receive the fastening devices 344 therethrough. In addition, the first body 340 includes one or more fastener receivers 348 formed thereon configured to receive and engage the fastening devices 344 therein, thereby coupling the first body 340 and the second body 342. The first body 340 and second body 342 cooperatively form a sensor receiving cavity 352 sized to receive a sensing device 354 therein.

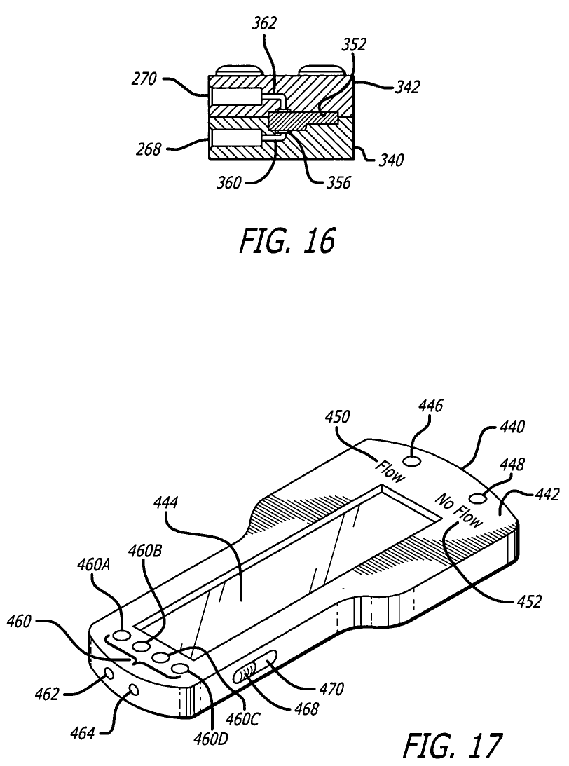

As shown in FIG. 16, the sensor receiving cavity 352 is in communication with the upstream flow port 268 formed on the body of the flow meter 240 through a first passage 360 formed in the first body 340. Similarly, the sensor receiving cavity 352 is in communication with the downstream flow port 270 formed on the flow meter 240 through a second passage 362 formed in the second body 342. Like the embodiment described above, the sensor device 354 may be sealed within the sensor housing 288 using one or more seals 356. The sensor device 354 is particularly configured to sense the pressure differential between the pressure of the upstream flow poor 268 and downstream flow port 270.

Referring back to FIG. 9, the output 214, flow path 220, upstream flow conduit 222, infusion conduit 230 and downstream flow conduit 232 may be manufactured in any variety of sizes and lengths as desired. In the preferred embodiment, these are manufactured of medical grade tubing. Further, the various connectors and couplers illustrated in FIG. 9 may be configured to detachably or nondetachably couple together the various elements illustrated in FIG. 9. In the preferred embodiment the infusion circuit is sterilized so that there is a sterile passageway for fluid expelled from the infusion device 212. Those in the art know various methods of sterilization for such devices.

During use, the infusion circuit 210 is primed in generally the same manner as the previous embodiment 10 (FIG. 1). Fluid flows from the infusion device 212 through the outlet 214, flow path 220, first restrictor 228, splitter 228, infusion circuit 230 and second restrictor 224 and out of the distal end 237. Referring also to FIGS. 1 and 2, in manner similar to the first conduit port 64 being placed in sensory communication with the pressure of the fluid at the monitor conduit port 26, upstream flow port 268 and downstream flow port 270 are place in sensory communication with the pressure of the fluid at the output 214 and splitter 228, respectively.

With upstream flow port 268 and downstream flow port 270 being placed in sensory communication with the pressure of the fluid at the output 214 and splitter 228, the fluid sensing device 354 of the flow meter 240 measures a differential pressure across the flow restrictor 224 within the infusion circuit 210. (See FIG. 9) More specifically, the pressure present within the infusion circuit 210 is measured at two locations: upstream of the flow restrictor 224, and downstream of the flow restrictor 224.

For a fully developed flow in a flow restrictor defining a passageway with a constant transverse dimension, the pressure decreases linearly from the entrance to the exit. As a result, the differential pressure between the inlet and outlet of the flow restrictor can be computed using the following equation:
ΔP=128 μLQ/πD⁴
where ΔP represents the differential pressure, μ represents the flow viscosity, L represents the length between the inlet and outlet of the flow restrictor, Q is the flow rate, and D is the diameter of the flow restrictor.

As stated above, the length and transverse dimensions of the flow restrictor 224 may be fixed. In addition, the viscosity of the fluid flowing through the flow restrictor may be approximated by the following equation:
μ=Be^{A/T}
where T represents the fluid temperature, while A and B are constants associated with the type of fluid flowing through the flow circuit. Any of the devices disclosed in the present application may include a temperature sensing device therein configured to measure the temperature of a fluid. Furthermore, the memory devices of the processing sections may be configured to store fluid temperature measurements. The relationship between pressure and flow rate may be expressed as follows:
   ΔP=KμQ=KBe^{A/T}Q=K′e^{A/T}Q
where K′ and A are constants that depend on the dimensions of the restrictor and the type of fluid flowing therethrough. In one embodiment, the constants associated with various drugs or therapeutic agents may be stored in a memory device coupled to the processing section 300. (See FIG. 11). As a result, the flow rate of a fluid flowing within the infusion circuit 210 may be determined based on the pressure measured therein and such determination may also include utilization of parameter supplied by other sensors or stored within the memory device.

During use, the flow rate may be expressed in a number of manners of the display 264 of the flow meter 240. For example, the flow rate may be expressed numerically or graphically. In addition, the flow meter 240 may further include a memory chip or other device attached to or otherwise in communication with the processing circuit illustrated in FIG. 4 and/or 12. For example, the memory device may comprise a erasable programmable read only memory (EPROM) chip configured to store the pressure or flow rate measured by the sensor 154 and/or sensor 354. (See FIGS. 7 and 15) Optionally, the flow information stored on the memory device may be either reviewed on the display 264 (if present) or downloaded to an external device. Exemplary external devices include, for example, computers, hand held PDA devices, or other systems configured to analyze data received from the flow meter 240. As such, the flow meter 240 may include one or more ports capable of receiving any number of connecting conduits therein. For example, the flow meter 240 may be configured to couple to external device through an RS 232 cable, an IR transmitter, or an RF transmitter.

In a further embodiment the flow meter 240 may utilize the timing device such that the rate of flow at predetermined time intervals may be measured and stored. The flow meter may then use the information to calculate and display the amount of fluid expelled from the device 212 which then flows through the infusion circuit 210 into the patient.

FIG. 17 shows an alternate embodiment of a flow meter and indicator for use with the infusion circuit 210 shown in FIG. 9. As shown, the flow meter and indicator 440 comprises a body 442 having at least one display 444 positioned thereon. In the illustrated embodiment, the display device 444 comprises a liquid crystal display configured to graphically present information to a user. Optionally, any number or type of display devices 444 may be used on the flow meter and indicator 440. For example, the display device 444 may include a plasma display device or a touch screen display. Referring again to FIG. 17, a first indicator 446 and a second indicator 448 are positioned proximate to the display device 444. In addition, a first legend 450 is positioned proximate to the first indicator 446. Similarly, a second legend 452 is positioned proximate to the second indicator 448. In the illustrated embodiment, a display control device 460 is positioned proximate to the display device 444 for controlling the presentation of information thereon. In the illustrated embodiment, four display control buttons 460A, 460B, 460C, and 460D are positioned on the body 442 proximate to the display device 444. In an alternate embodiment, the display control 460 may include any number or type of control devices including, without limitation, buttons, wheels, finger pads, keys, or track control balls.

Referring once again to FIG. 17, the body 442 includes an upstream conduit port 462 and a downstream conduit port 464 formed thereon. Optionally, an activation switch 468 may be positioned within an activation switch recess 470 formed on the body 442 of the flow meter and indicator 440.

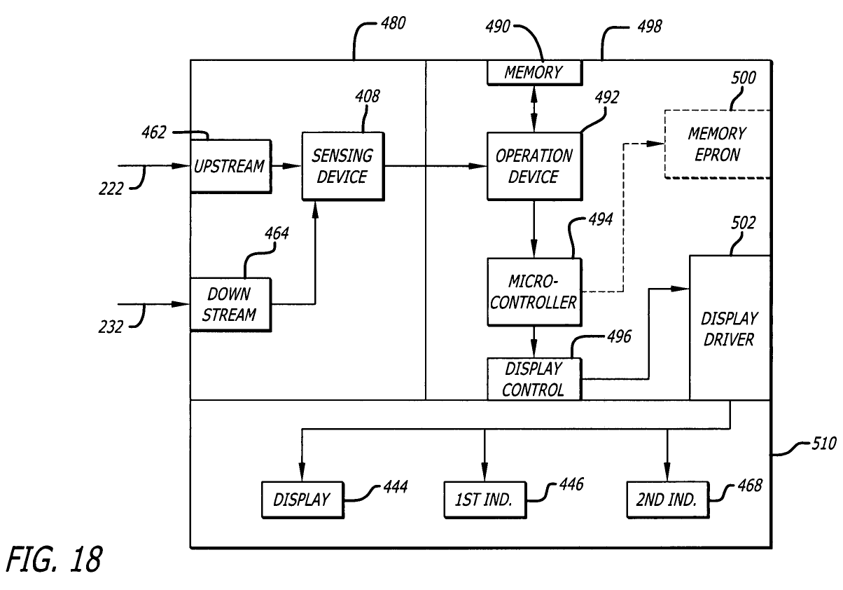

FIG. 18 shows a block diagram of a control circuit for use within the flow meter and indicator 440. In one embodiment, the flow meter and indicator 440 may be used with infusion circuit 210 shown in FIG. 9. As shown in FIG. 18, the control circuit includes a sensor section and housing 480, a processing section 498, and a display section 510. The sensor section and housing 480 includes an upstream flow port 462 that is in communication with an upstream flow conduit 222 connected to the infusion circuit 210. Similarly, the downstream flow port 464 is connected to the downstream flow conduit 232 positioned downstream of the flow restrictor 224. The sensing device 488 receives inputs from the upstream flow port 462 and the downstream flow port 464. The sensing device 488 is in communication with an operational device 492 of the processing section 498. The operational device 492 may be in communication with a memory device 490 positioned within the processing section 498. Exemplary memory devices include deproms, flash cards, or other information storage devices.

Like the previous embodiments, the operational device 492 receives information from the sensing device 488, processes the information, and sends the information to a microcontroller 494 positioned within the processing section 498. The microcontroller 494 processes the information and sends display information to a display controller 496 that sends the display signal to a display driver 502 in communication therewith. The display driver 502 processes the information received from the display controller 496 and sends a display signal to the appropriate display device. For example, the display driver 402 may send a signal to the display device 444 located on the body 442 of the flow meter and indicator 440. In addition, the display driver 402 is configured to control the operation of the first indicator 446 and the second indicator 468.

Like the previous embodiment, the flow meter and indicator 440 utilizes a sensor section and housing 480 shown in FIGS. 13-16 and described above. During use, the flow meter and indicator 440 performs multiple functions. For example, the flow meter and indicator 440 utilizes a first and second indicator 466, 468, respectively, to alert a user to the presence of a flow through a flow circuit 210 by comparing the pressure within the flow circuit 210 to ambient pressure. In addition, the flow meter and indicator 440 is configured to display flow rate information on the display 444. Therefore, the flow meter and indicator 440 combines the benefits of the previous embodiments in to a single device. Like the previous embodiments, any variety of indicators and display devices may be used with the present embodiment. Exemplary indicators include, without limitation, light emitting diodes, incandescent bulbs, fuses, or similar devices. Likewise, exemplary display devices include, without limitation, liquid crystal displays, plasma displays, and touch screen displays.

As the infusion circuit 210 is used to introduce fluids intravenously into a patient, the device should be sterilized and properly packaged. All passageways through which fluid flows or is exposed to should be sterilized and maintained in a sterile manner prior to use.

Embodiments disclosed herein are illustrative of the principles of the invention. Other modifications may be employed which are within the scope of the invention; thus, by way of example but not of limitation, alternative coupling devices, alternative infusion devices, and alternative electronic components. Accordingly, the devices disclosed in the present application are not limited to that precisely as shown and herein.

1. A flow indicating infusion set for an ambulatory infusion pump, the set comprising:
an infusion circuit configured to be placed in fluid communication with the pump, the infusion circuit the circuit defining a passageway for fluid expelled from the pump, the circuit including a first flow restrictor and a second flow restrictor;
a flow indicator device having a body, the body having first conduit port and a second conduit port formed thereon;
at least one indicator conduit providing sensory communication between the first conduit port and a fluid pressure present at a location between the first flow restrictor and the second flow restrictor;
a sensing device positioned within the body and in fluid communication with the first and second conduit ports, to sense the pressure differential between the pressures present at the first and second conduit ports, the sensing device configured to output a signal in dependence on the sensed pressure; and
at least one indicator positioned on the body and in communication with the sensing device to receive the signal.

2. The device of claim 1 wherein the sensing device further comprises:
a sensor housing defining a sensor device cavity, the sensor device cavity in fluid communication with the first and second conduit ports; and
a sensor positioned with the sensor device cavity.

3. The device of claim 2 wherein the sensor comprises a piezoelectric sensor.

4. The device of claim 2 wherein the sensor comprises a low flow pressure sensor.

5. The device of claim 1 wherein the second conduit port is in sensory communication with the pressure of the atmosphere.

6. The device of claim 1 wherein the at least one indicator comprises a light emitting diode.

7. The device of claim 1 wherein the at least one indicator comprises an audible alarm.

8. The device of claim 1 wherein the pump is an elastomeric pump.

9. The device of claim 1 wherein the passageway is sterilized.

10. A flow measuring infusion set for an ambulatory infusion pump, the set comprising:
a flow circuit configured to be placed in fluid communication with the pump, the flow circuit defining a passageway for fluid expelled from the pump, the circuit including a flow restrictor disposed along the length thereof;
a flow meter body disposing at least one display device thereon, the flow meter body including an upstream flow port and a downstream flow port;
an upstream flow conduit providing sensory communication between the upstream flow port and a fluid pressure present at a location upstream of the flow restrictor;
a upstream flow port formed on the flow meter body and in sensory communication with the upstream flow conduit;
a downstream flow conduit providing sensory communication between the upstream flow port and a fluid pressure present at a location downstream of the flow restrictor;
a sensing device positioned within the flow meter body and in communication with the upstream flow port and the downstream flow port, the sensing device in communication with the display device.

11. The device of claim 10 further comprising a sensor housing having a sensor receiving cavity formed therein, the sensor receiving cavity in communication with the upstream flow port and the downstream flow port.

12. The device of claim 11 further comprising a pressure differential sensor positioned within the sensor housing.

13. The device of claim 10 wherein the display device is a liquid crystal display.

14. The device of claim 10 wherein the sensor comprises a piezoelectric sensor.

15. The device of claim 10 wherein the sensing device is configured to measure flow rates of about 0.1 ml/hr to about 2 ml/hr.

16. The device of claim 10 wherein the sensing device includes means for calculating the volume of fluid that has flowed through the restrictor

17. The device of claim 10 further comprising a temperature sensor in communication with the infusion circuit and the sensing device, the temperature sensor configured to measure the temperature of a fluid within the infusion circuit.

18. The device of claim 17 further comprising a timing device positioned therein and configured to measure the rate of a flow through the circuit as a function of time.

19. A device for measuring the flow rate of fluid expelled by a pump through an infusion circuit, the circuit including a flow restrictor, the device comprising:
a body disposing at least one display device and at least one indicator thereon;
an upstream flow conduit in communication with a flow circuit upstream of the flow restrictor;
a upstream flow port formed on the body and in fluid communication with the upstream flow conduit;
a downstream flow conduit in communication with a flow circuit downstream of the flow restrictor;
a downstream flow port formed on the body and in fluid communication with the downstream flow conduit;
a sensing device positioned within the flow indicator body and in fluid communication with the upstream flow port and the downstream flow port, the sensing device in communication with the display device and the indicator; and
an timing device in communication with the flow measuring device and configured to measure the total flow volume within a flow circuit by measuring the flow rate flowing through the infusion circuit as a function of time.
